(19) United States
(12) Patent Application Publication
(10) Pub. No.: US 2023/0028742 A1
(43) Pub. Date: Jan. 26, 2023

(54) COMPOSITIONS AND USES THEREOF

(71) Applicant: The University of Melbourne, Melbourne (AU)

(72) Inventors: Garth CAMERON, Melbourne (AU); Nicholas Anthony GHERARDIN, Melbourne (AU); Catarina Filipa DOS SANTOS SA E ALMEIDA, Melbourne (AU); Dale Ian GODFREY, Melbourne (AU)

(21) Appl. No.: 17/788,156

(22) PCT Filed: Dec. 24, 2020
(86) PCT No.: PCT/AU2020/051433

§ 371 Date: Jun. 22, 2022

(30) Foreign Application Priority Data
Dec. 24, 2019 (AU) 2019904920
Dec. 24, 2019 (AU) 2019904921

(51) Int. Cl. C07K 16/28; C12N 5/0784; A61P 37/06

(52) U.S. Cl. CPC C07K 16/2896; C12N 5/0639; A61P 37/06

(57) ABSTRACT

The present disclosure relates generally to compositions for inducing an immunosuppressive phenotype in an antigen presenting cell of the immune system via ligands that activate CD1d signaling. Uses thereof in a therapeutic protocol for treating or preventing conditions associated with aberrant immune system activation, such as autoimmune disease, inflammatory disease, allergy and transplant rejection are detailed.

### RELATED APPLICATIONS

This application claims priority from Australian Provisional Patent Application Nos. 2019904920 and 2019904921 filed on 24 Dec. 2019, the entire contents of which are hereby incorporated by reference.

### FIELD

The present disclosure relates generally to compositions for inducing an immunosuppressive phenotype in a cell of the immune system and uses thereof in a therapeutic protocol for treating or preventing conditions associated with aberrant immune system activation.

### BACKGROUND

The immune system has the ability to induce profound effects, both beneficial and detrimental, in a subject. The regulation of the immune system is highly complex, especially due to the pleiotropic nature of cytokines released as part of an immune response. The identification of effective regulators of immune function has been a major goal of researchers for decades. Such regulators are needed to either promote or abate the immune response in a directed manner, depending on a disease or condition being treated and the manner of treatment.

Immunotherapy has emerged as a major therapeutic option for the treatment of diseases such as cancer, autoimmune diseases, infection, allergy, allograft rejection and hypersensitivity. Such immunotherapeutic treatments can help boost the immune system, enabling it to, for example, successfully fight off cancer cells. By contrast, in diseases caused by an overactive immune system, immunotherapy can be used to specifically dampen or suppress the abnormal immune response to treat the underlying cause of the disease and reduce symptoms.

Despite significant advances in the development of effective immunotherapies, regulating the immune system is complex and significant obstacles still exist in the field of immunotherapy. These include the ability to identify effective regulators of immune function that can be modulated in a predictable manner that results in an effective and safe patient response.

There remains, therefore, an urgent need for improved methods of suppressing an immune response in a manner that is adapted for treatment or prevention of conditions associated with aberrant immune system activation, for example, autoimmune diseases, inflammatory diseases and allergy.

### SUMMARY

The present disclosure identifies the cluster of differentiation 1 (CD1) family of molecules as effective targets for modulation of the immune system. Taught herein is a group of immunomodulators that are selected to bind to CD1 thereby suppressing an immune response.

Accordingly, in an aspect disclosed herein there is provided a method for suppressing an immune response in a subject, the method comprising administering an effective amount of a composition comprising a ligand that activates signalling through a CD1 molecule expressed by an antigen-presenting cell (APC) in the subject.

In another aspect disclosed herein, there is provided a method for inducing an immunosuppressive phenotype in an APC, said method comprising contacting the APC with an effective amount of a composition comprising a ligand that activates signalling through a CD1 molecule expressed by the APC.

The present disclosure also extends to pharmaceutical compositions for use in methods for suppressing the immune system.

In an aspect, the present disclosure provides the use of a pharmaceutical composition comprising a ligand that activates signalling through a CD1 molecule expressed on an APC in the manufacture of a medicament for the treatment of an autoimmune disease, an inflammatory disease or allergy. In an embodiment, the inflammatory disease is transplant rejection or graft-versus-host disease.

In another aspect, there is provided a pharmaceutical composition comprising a ligand that activates signalling through a CD1 molecule expressed on an APC for use in the treatment of an autoimmune disease, an inflammatory disease or allergy in a subject. In an embodiment, the inflammatory disease is transplant rejection or graft-versus-host disease.

In another aspect, there is provided a composition comprising a ligand that activates signalling through a CD1 molecule expressed on an APC for use in inducing an immunosuppressive phenotype in the APC.

In another aspect, there is provided a method for suppressing an immune response in a subject, the method comprising:
a. isolating APCs from the peripheral blood of a donor;
b. culturing the isolated APCs with a composition comprising a ligand that activates signalling through CD1 molecules expressed on the APCs for a time and under conditions suitable to induce an immunosuppressive phenotype in the APCs; and
c. administering the APCs with an immunosuppressive phenotype to the subject,
   wherein the APCs with an immunosuppressive phenotype suppress an immune response in the subject.

In another aspect, there is provided a method identifying CD1-restricted T cells from a plurality of immune cells, the method comprising:
a. obtaining a plurality of immune cells from the peripheral blood of a donor;
b. contacting the plurality of immune cells with a composition comprising a first ligand, wherein the first ligand blocks the interaction between a CD1 molecule and a CD36 family member;
c. contacting the plurality of immune cells with second ligand, wherein the second ligand is capable of binding a CD1-restricted TCR; and
d. identifying CD1-restricted cells, wherein the CD1-restricted cells comprise second ligand bound to the cell surface.

### BRIEF DESCRIPTION OF THE DRAWINGS

FIG. 1 shows FACS plots showing that CD1 tetramers bind to non-T cells in human blood. (A) A graphical representation of the proportion of CD3+ cells (x-axis) bound to CD1 tetramers that are loaded with undefined, endogenous (endo) lipid antigens (i.e. CD1a-endo, CD1b-endo, CD1c-endo, CD1d.endo). CD1d loaded with α-Galactosylceramide lipid antigen is also shown as CD1d-αGalCer and MR1 loaded with 5-(2-oxopropylideneamino)-6-D-ribitylaminouracil (5-OP-RU) vitamin B metabolite antigen is shown as MR1-5-OP-RU which highlights the brightly staining population of CD1d-αGalCer-specific Natural Killer T (NKT) cells and MR1-5-OP-RU-specific MAIT cells respectively. Streptavadin-BV421 (SAv-BV421) is shown as a negative control for staining. For CD1-endo tetramers, black show staining on CD3+ T cells, grey boxes show staining on CD3⁻ non-T cells. Black arrows indicate what appears to be TCR-independent CD1 tetramer staining. (B) A graphical representation of CD1 tetramer staining (different tetramers along x-axis) and the % of CD1 tetramer positive cells (%), or the mean fluorescence intensity (MFI) of CD1 tetramer staining (y-axis) as measured in lymphocytes (top row), monocytes (middle row) and platelets (bottom row). Each dot represents a different blood sample from different donor (n=20 blood samples from separate donors).

FIG. 2 shows that CD1c tetramers bind via their α1/α2 domains. Flow cytometry plots showing gating strategy for lymphocytes, divided into CD3+ T cells or CD19+ B cells. On the right, gated CD3+ T cells (top row), CD19+ B cells (middle row) and CD14+ monocytes (bottom row) cells were stained with CD1b tetramer, CD1c tetramer or a hybrid CD1c/CD1b tetramer where the α1/α2 domain is from CD1c and the α3 domain is from CD1b. Data is representative of 2 independent experiments.

FIG. 3 shows that CD1c tetramers bind to non-T cell lines in vitro. Flow cytometry data depicting cell line staining with CD1c-endo tetramer or CD1d-endo tetramers (y-axis; count (modal); x-axis CD1 tetramer staining) of C1R (B cell line), MEG-01 (megakaryocyte), THP-1 (monocyte), K562 (myeloid leukaemia), Jurkat (T cell) and MOLT4 (T cell) cell lines. Data is representative of 2 independent experiments.

FIG. 4 shows that CD36L1 is a ligand for CD1c. (A) Flow cytometry data depicting C1R parental cells (top panel) and C1R CRISPR/Cas9 genome-wide deletion library cells (bottom panel) used to investigate the ligand responsible for CD1c-endo tetramer binding. The percentage of unstained cells is depicted in each plot. (B) A graphical representation of genes targeted by guide RNAs in CD1c tetramer negative cells (derived from the CD1c tetramer-cells in sort 2 of (A) as compared to unsorted (CD1c tetramer+) C1R cells. Data points within boxes represent sgRNAs corresponding to SCARB1 (CD36L1). Grey data points represent sgRNAs overrepresented (>0 log 2(fold-change)) or underrepresented (<0 log 2(fold-change)) in the enriched pool. For clarity, underrepresented data points <0 log 2(fold-change) are greyed out. (C) Tabulated data gene expression data identifying deleted genes that might be responsible for CD1c tetramer reactivity. Arrows indicating SCARB1 (CD36L1) sgRNAs.

FIG. 5 shows that CD36 and CD36L1 can both bind to CD1 tetramers. (A, left panel) A flow cytometry-based graphical representation of C1R cells stained with anti-CD36L1 (x-axis) and/or CD1c-endo tetramer (y-axis). The population labelled ‘4’ depicts unstained cells, ‘3’ depicts cells stained with CD1c-endo alone, ‘2’ with anti-CD36L1 alone and ‘1’ with dual staining; (A, right panel) a flow cytometry-based graphical representation of Förster Resonance Energy Transfer (FRET) (x-axis) as a reflection of the proximity of CD36-L1 and CD1c tetramer on the surface of C1R cells (y-axis; counts (modal)). Data representative of 2 independent experiments. (B) A flow cytometry-based graphical representation of the expression of CD1c-endo tetramer (1^{st} column); CD36 (2^{nd} column) and CD36L1 (i.e., SCARB1) (3^{rd} column) (y-axis) versus CD3 or CD14 (x-axis) on platelets (top row), monocytes (middle row) and lymphocytes (bottom row). (C)(i) A flow cytometry based graphical representation of HEK293T cells stained with CD1c-endo tetramer (y-axis) and/or anti-CD36L1 (x-axis). The population labelled ‘4’ depicts unstained cells, ‘3’ depicts cells stained with CD1c-endo alone, ‘2’ with anti-CD36L1 alone and ‘1’ with dual staining. (C)(ii) A flow cytometry-based graphical representation of anti-CD36L1 staining (x-axis) in CD36L1 knockout HEK293T cells (top) and HEK293T wildtype (middle) cells and no-antibody control of HEK293t wildtype cells (bottom). Data representative of 2 independent experiments. (D) A flow cytometry-based graphical representation of CD1a, CD1b, CD1c or CD1d tetramer staining (y-axes) of CD36L1 knockout HEK293t cells (from C(ii)) transfected with either CD36 (top row), CD36L1 (middle row) or CD36L2 (bottom row). Data representative of 3 independent experiments. (E) A flow cytometry-based graphical representation of inhibition of CD1c tetramer staining (y-axis) in CD3+ and CD3-PBMC populations (x-axis) following pre-incubation with a range of doses of anti-CD36 blocking antibody. (F) A flow cytometry-based graphical representation of CD1c tetramer staining of CD3+ and CD3− lymphocytes (top row), CD14+ monocytes (middle row) and CD42b+ platelets (bottom row) in the presence (right panel) or absence (left panel) of an anti-CD36 blocking antibody. The few remaining CD1c tetramer+ lymphocytes (within region of top middle panel) were highly enriched for γδ T cells as shown by TCRγδ staining of this population (right panel). Data depicts 1 representative donor from a cohort of 12 donors.

FIG. 6 shows that CD1a and CD1c ligation drives PD-L1 and PD-L2 upregulation. (A) Left panels show flow cytometry-based graphical representations of PD-L1 (top two rows) and PD-L2 expression (bottom two rows) by human Monocyte-derived-DC (Mo-DCs) cultured with plate-bound anti-CD1a, anti-CD1c, or isotype control antibody in the presence or absence of low dose (1 ng/ml) lipopolysaccharide (LPS). Graph depicts the percentages of PD-L1 high and PD-L2 positive Mo-DCs following 48 hr culture under the indicated conditions (right). Data pooled from 8 independent experiments (n=4 donors). (B) A graphical representation of dual PD-L1+/PD-L2+ cells, or IL-12p40 or IL-8 cytokine concentrations (y-axis) from Mo-DCs following stimulation with anti-CD1a, anti-CD1c and isotype control antibody in the presence (grey bars) or absence (white bars) of low dose LPS. Data pooled from 4 independent experiments (n=4 donors). (C) A graphical representation of IL-8, IL-12p40, IL-12p′70 and IL-23 cytokine concentration (y-axis; pg/ml) produced by Mo-DCs following stimulation with anti-CD1a, anti-CD1c and isotype control antibody (x-axis) in the presence of low dose LPS (1 ng/ml). Cytokine analysis performed on 2 independent experiments (n=2 donors). (D) A graphical representation of the correlation between IL-12p40 concentrations (y-axis; pg/ml) and the percentage of Mo-DCs co-expressing PD-L1 and PL-L2 (x-axis). Data pooled from 4 independent experiments (n=4 donors).

FIG. 7 shows the specificity of PD-L1 and PD-L2 induction on Mo-DCs following CD1a or CD1c ligation. A flow cytometry-based graphical representation of the proportion of Mo-DCs expressing high levels of PD-L1 (top row) and PD-L2 (bottom row) following stimulation with anti-CD1a, anti-CD1c, anti-CD40, anti-CD11c, anti-HLA-DR and isotype control antibody, in the presence of low dose LPS (1 ng/ml). Representative of 2 independent experiments (n=3 donors).

FIG. 8 shows PD-L1 and PD-L2 upregulation by Mo-DCs cultured in the presence of anti-CD1a, or anti-CD1b, or anti-CD1c, or an isotype control, or no antibody. Each culture also contained low dose cytokines IL-1β and TNF as costimulatory factors. Representative of 5 similar experiments (n=10 donors).

FIG. 9 shows that CD36-blockade facilitates ex vivo analysis of CD1d-restricted NKT cells and CD1b-glucose-monomycolate (GMM)-reactive germline-encoded mycolyl lipid reactive (GEM)-T cells. (A) A series of graphical representations of FACS plots from 3 healthy donor PBMC samples showing CD1d tetramer staining on CD3+ T cells where CD1d tetramer was loaded with endogenous lipid antigen, or lysophosphatidylcholine (LPC), or sulfatide, or α-galactosylceramide, with and without CD36-blockade. (B) A series of graphical representations of FACS plots from 3 healthy donor PBMC samples showing CD1b-GMM tetramer staining on CD4+ T cells with and without CD36-blockade.

FIG. 10 shows that CD1 cross-linking downregulates Fc receptors (CD16) on Mo-DC cultures in the presence of IL-1β and TNF. (A) A series of graphical representations of CD16 (Fc receptors FcγRIIIa (CD16a) and FcγRIIIb (CD16b); y-axis) and CD64 (Fc-gamma receptor 1 (FcγR1; x-axis) expression following CD1 cross-linking as measured by flow cytometry. (B) (top) A graphical representation of PD-L2 versus CD16 staining of MoDCs following stimulation with anti-CD1c or isotype control, as measured by flow cytometry; (bottom) A graphical representation of the proportion of PD-L2+ CD16− Mo-DCs (%; y-axis) following incubation with anti-CD1c as compared to isotype control (x-axis). Each data point represents a different donor.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The singular forms “a”, “an” and “the” include plural aspects unless the context clearly dictates otherwise. Therefore, for example, reference to “a cell” includes a single cell, as well as two or more cells; reference to “an agonist” includes a single agonist, as well as two or more agonists.

The present disclosure is predicated, at least in part, on the unexpected finding that CD1 molecules on the surface of antigen-presenting cells (APCs), e.g., dendritic cells, can bind to unexpected and widely expressed ligands being members of the CD36 family. The present disclosure is also predicated, at least in part, on the unexpected finding that ligation of CD1 molecules can induce an immunosuppressive phenotype in the APCs that express these CD1 molecules. In an embodiment, agents that bind to CD1 molecules, such as CD36 proteins or anti-CD1 antibodies, can be used, for example, to treat or prevent conditions associated with aberrant immune system activation, e.g., an autoimmune condition or transplant rejection.

Accordingly, the present disclosure teaches a method for suppressing an immune response in a subject, said method comprising administering an effective amount of a composition comprising a ligand that activates signalling through a CD1 molecule expressed by an APC in the subject.

The term “CD1 molecule” includes but is not limited to, in humans, the MHC-class-I-like glycoproteins CD1a, CD1b, CD1c, CD1d and CD1e. CD1a, CD1b, CD1c and CD1d are expressed on the cell membrane, while CD1e is expressed intracellularly (Godfrey et al., 2015, Nature Immunology, 16: 1114-1123). By contrast, mice only have the CD1d gene (Godfrey et al., 2015, supra). Homologs and variants of CD1 molecules in other mammals are also contemplated herein depending on the subject being treated.

CD1 isoforms can be classified into three groups: Group I comprises CD1a, CD1b and CD1c; Group II comprises CD1d; and Group III comprises CD1e (Godfrey et al., 2015, supra).

In an embodiment, the CD1 molecule is a Group I CD1 molecule selected from the group consisting of CD1a, CD1b and CD1c. In another embodiment, the Group I CD1 molecule is CD1c.

In an embodiment, the CD1 molecule is CD1d.

CD1 molecules are typically characterised as antigen-presenting molecules. These proteins present lipid-based antigens to CD1-restricted T cells via specific T cell receptors. Group I CD1 molecules mainly present lipid antigens to clonally diverse T cells that mediate adaptive immunity to the vast range of microbial lipid antigens (Godfrey et al., 2015, supra). By contrast, Group II CD1 molecules present lipid antigens to natural killer T (NKT) cells, a subset of which, sometimes known as “type-I” or “invariant NKT (iNKT)” cells, express an invariant T-cell receptor (TCR) α-chain.

The term “immune response” generally refers to a biological response to a stimulus that is perceived to be adverse and/or foreign, such as bacteria and viruses. Suitable immune responses will be known to persons skilled in the art, illustrative embodiments of which include adaptive and innate immune responses Immune responses can also be characterised as humoral responses and/or cellular responses, or as immunosuppressive or immunopotentiating.

The immune response referred to herein does not need to manifest as a measurable/detectable symptom or effect. An immune response may be characterised by the release of immunomodulatory mediators (i.e., immunostimulatory and/or immunosuppressive mediators) that will be familiar to persons skilled in the art, illustrative examples of which include cytokines (e.g., IFN-γ, TNF, IL-1, IL-2, IL-4, IL-8, IL-10, IL-12, TGF-β), growth factors, plasma-derived mediators (e.g., Bradykinin, C3, C5a, Factor XII, membrane attack complex, plasmin, thrombin), chemokines (e.g., CCL5, CCL17), monoamines, and eicosanoids (e.g., leukotriene B4, LTC4, LTD4, 5-oxo-eicosatetraenoic acid, 5-HETE, prostaglandins).

In an embodiment, an immunosuppressive response is characterised by the release of one or more immunomodulatory mediators such as IL-8 and IL-12p40 (monomers or dimers) IL-10, and TGF-β.

The term “signalling” as used herein refers to the transmission of signals following binding to a CD1 molecule and/or crosslinking of CD1 molecules on the cell surface, illustrative examples of signalling include IL-2 signalling to stimulate naïve CD4+T cell proliferation, and IFN-γ signalling to promote the expression of MHC molecules on antigen-presenting cells.

As disclosed elsewhere herein, binding to CD1 and/or the crosslinking of CD1 results in the induction of an immunosuppressive phenotype. An immunosuppressive phenotype will be familiar to persons skilled in the art, illustrative examples of which include the upregulation of PD-L1 and/or PD-L2, and the production of immunoregulatory cytokines (e.g. IL-12p40 (monomers or dimers) and/or IL-8), which can inhibit immunity in vivo.

Methods for determining whether a composition or agent can induce an immunosuppressive phenotype in APCs in accordance with the present invention would be known to persons skilled in the art, illustrative examples of which are referred to or described in Hancock et al. (2013, Clinical and Experimental Immunology, 171(2): 147-154), the entire contents of which is incorporated herein by reference. Other suitable assays for determining whether a composition or agent can induce an immunosuppressive phenotype in APCs in accordance with the present invention include killing assays, proliferation assays, in vitro mixed lymphocyte reaction, and MAIT cell proliferation assay.

In an embodiment, the activation of signalling through the CD1 molecule expressed by the APC in the subject is characterised by upregulation of the expression of one or more molecules selected from the group consisting of PD-L1, PD-L2, IL-12p40 and IL-8.

The terms “antigen-presenting cell” and “APC” are used interchangeably herein to refer to a cell that mediates the immune response by processing and presenting antigens for recognition by lymphocytes, such as T cells. Suitable APCs would be known to persons skilled in the art, illustrative examples of which include dendritic cells, macrophages, monocytes, Langerhans cells and B cells.

The term “antigen” as used herein refers to a molecule that is capable of stimulating an immune response. Each antigen comprises distinct epitopes, which may result in specific immunological responses. Suitable antigens would be known to persons skilled in the art, illustrative examples of which include peptide antigens recognised by MHC restricted CD4 and CD8 T cells, lipid antigens (e.g., lipid antigens presented to NKT cells by CD1d), MR1-restricted antigens (e.g., MR1-restricted vitamin-B2 derivatives including 5-OP-RU, or other vitamin B derivatives presented to mucosal-associated invariant T (MAIT) cells).

In an embodiment, the APC is a professional APC.

Professional APCs are immune cells that specialise in presenting an antigen to a T cell. In an embodiment, a professional APC takes up a protein antigen, processes it into peptide antigens, and returns the peptide antigens to the surface of the cells in complex with class I or class II major histocompatibility complex (MHC) molecules. The T cell is activated when it interacts with the formed peptide-MHC complex.

In another embodiment, a professional APC takes up a lipid-based antigen, and returns it to the surface of the cell in complex with CD1 antigen-presenting molecules. CD1-lipid-reactive T cells are activated when they interact with the formed lipid-CD1 complex.

In another embodiment, a professional APC takes up an MR1-restricted antigen, and returns it to the surface of the cell in complex with MR1 antigen-presenting molecules. MAIT cells are activated when they interact with the formed MR1-Ag complex.

Suitable professional APCs will be known to persons skilled in the art, illustrative examples of which include dendritic cells, macrophages and B cells.

In an embodiment, the APC is a dendritic cell. In another embodiment, the APC is a monocyte-derived dendritic cell (Mo-DC).

In an embodiment, the APC is a non-professional APC.

Suitable non-professional APCs will be known to persons skilled in the art, illustrative examples of which include stromal cells expressing CD1c in an inflammatory setting.

As used herein, the term “suppression” and variations thereof such as “suppressed” and “suppressing” do not necessarily imply the complete suppression of the specified immune response. Rather, the suppression may be to an extent, and/or for a time, sufficient to produce the desired effect. Suppression may be prevention, retardation, reduction or otherwise hindrance of the immune response. Such suppression may be in magnitude and/or be temporal in nature. In particular contexts, the terms “suppress” and “inhibit,” and variations thereof may be used interchangeably.

The term “subject” as used herein refers to mammals and includes humans, primates, livestock animals (e.g., sheep, pigs, cattle, horses, donkeys), laboratory test animals (e.g., mice, rabbits, rats, guinea pigs), companion animals (e.g., dogs, cats) and captive or wild animals (e.g., foxes, kangaroos, deer). Typically, the mammal is human, laboratory test animal or companion animal More typically, the mammal is a human.

In an embodiment, the subject has, or is at risk of developing, a condition characterised by activation of their immune system, illustrative examples of which include an autoimmune disease, inflammatory disease, transplant rejection, graft-versus-host disease and allergy.

In an embodiment, the subject has a condition selected from the group consisting of an autoimmune disease, an inflammatory disease and allergy.

Suitable autoimmune diseases would be known to persons skilled in the art, illustrative examples of which include systemic lupus erythematosus, rheumatoid arthritis, spondyloarthritis, diabetes, polychondritis, psoriasis, dermatitis, inflammatory bowel disease, Crohn's disease, ulcerative colitis, coeliac disease, meningitis, multiple sclerosis and asthma.

Suitable inflammatory diseases would be also be known to persons skilled in the art, illustrative examples of which include asthma, chronic peptic ulcer, tuberculosis, arthritis, periodontitis, ulcerative colitis, Crohn's disease, sinusitis, active hepatitis, cancer and atherosclerosis. In an embodiment, the inflammatory disease is transplant rejection or graft-versus-host disease.

In an embodiment, the subject is a transplant recipient.

The term “transplant” refers to an organ, tissue or cell that has been transplanted from one subject to a different subject, or transplanted within the same subject (e.g., to a different area within the subject). Organs such as liver, kidney, heart or lung, or other body parts, such as bone or skeletal matrix such as bone marrow, tissue, such as skin, intestines, endocrine glands, or stem cells or various types, or hematopoietic cells including hematopoietic stem and progenitor cells, are all examples of transplants. The transplant can be an allograft, autograft, isograft, or xenograft. The term “allograft” refers to a graft between two genetically non-identical members of a species. The term “autograft” refers to a graft from one area to another on a single individual. The term “isograft” or syngraft” refers to a graft between two genetically identical individuals. The term “xenograft” refers to a graft between members of different species.

In an embodiment, the ligand comprises a peptide, an antibody or antigen-binding fragment thereof, or a non-peptide molecule.

In an embodiment, the ligand is an antibody or an antigen-binding fragment thereof. Such antibodies, or antigen-binding fragments thereof, may be of any type, such as a murine (e.g., mouse or rat), a chimeric, a humanised or a human antibody. A “human” antibody (including an antigen-binding fragment thereof) is typically defined as one that is not chimeric (e.g., not “humanised”) and not from (either in whole or in part) a non-human species. A human antibody can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analysing a database of human antibody or antibody fragment sequences and devising a polypeptide sequence utilising the data obtained therefrom. Another example of a human antibody or functional antibody fragment is one that is encoded by a nucleic acid isolated from a library of antibody sequences of human origin (i.e., such a library based on antibodies taken from a human natural source).

A “humanised antibody” or humanised antigen-binding fragment thereof is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin.

The term “chimeric antibody” or functional chimeric antibody fragment is defined herein as an antibody molecule that has constant antibody regions derived from, or corresponding to, sequences found in one species and variable antibody regions derived from another species. Generally, the constant antibody regions are derived from, or corresponding to, sequences found in humans, e.g., in the human germ line or somatic cells, and the variable antibody regions (e.g., VH, VL, CDR or FR regions) are derived from sequences found in a non-human animal, e.g., a mouse, rat, rabbit or hamster.

Also, as used herein, an “immunoglobulin” (Ig) hereby is defined as a protein belonging to the class IgG, IgM, IgE, IgA, or IgD (or any subclass thereof), and includes all conventionally known antibodies and functional fragments thereof. A “functional fragment” of an antibody/immunoglobulin is defined as a fragment of an antibody/immunoglobulin (e.g., a variable region of an IgG) that retains the antigen-binding region. An “antigen-binding region” or “antigen-binding fragment” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs. One class of immunoglobulins for use in the present invention is IgG. “Functional fragments” include the domain of a F(ab′)₂ fragment, a Fab fragment, scFv or constructs comprising single immunoglobulin variable domains or single domain antibody polypeptides, e.g., single heavy chain variable domains or single light chain variable domains. The F(ab′)₂ or Fab may be engineered to minimise or completely remove the intermolecular disulphide interactions that occur between the C_{H1} and C_{L} domains. It is proposed that these fragments can activate CD1.

An antibody described herein may be derived from a recombinant antibody library that is based on amino acid sequences that have been designed in silico and encoded by nucleic acids that are synthetically created. In silico design of an antibody sequence is achieved, for example, by analysing a database of human sequences and devising a polypeptide sequence utilising the data obtained therefrom. Methods for designing and obtaining in silico-created sequences are described, for example, in Knappik et al., 2000, Journal of Molecular Biology, 296:57; Krebs et al., 2001, Journal of Immunological Methods, 254:67; Rothe et al., 2008, Journal of Molecular Biology, 376:1182 and U.S. Pat. No. 6,300,064.

In an embodiment, the ligand is an antibody to CD1 or an antigen-binding fragment thereof.

Methods of making antigen-specific binding agents, including antibodies and their derivatives and analogs and aptamers, are well known in the art. Polyclonal antibodies can be generated by immunisation of an animal Monoclonal antibodies can be prepared according to standard (hybridoma) methodology. Antibody derivatives and analogs, including humanised antibodies can be prepared recombinantly by isolating a DNA fragment from DNA encoding a monoclonal antibody and subcloning the appropriate V regions into an appropriate expression vector according to standard methods. Phage display, yeast display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Yeast display is described for example and without limitation in U.S. Pat. Nos. 6,423,538 and 9,139,637. Aptamer technology is described for example and without limitation in U.S. Pat. Nos. 5,270,163; 5,475,096; 5,840,867 and 6,544,776.

The terms “variant” and “derivative” in relation to an antibody or a CD1 molecule or its ligand refer to an amino acid sequence displaying substantial sequence identify or similarity with a reference amino acid sequence. The terms “variant” and “derivatives” also includes naturally occurring allelic variants.

A “derivative” also includes a mutant, fragment, part, portion or hybrid molecule with reference to an antibody or CD1 molecule. A derivative generally but not exclusively carries a single or multiple amino acid substitution, addition and/or deletion.

A “homolog” includes an analogous polypeptide having at least about 80% similar amino acid sequence from another animal species or from a different locus within the same species.

A variant also includes an “analog” which is generally a chemical analog. Chemical analogs of a CD1 molecule contemplated herein include, but are not limited to, modification to side chains, incorporation of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Such analogs may be useful in synthetic vaccines to generate antibodies for use as targeting agents. Analogs may have attributes such as increased serum half-life. The antibodies may also be in anti-sera that comprise CD1-binding antibodies or antibodies that bind other CD1 binding ligands.

In an embodiment, the ligand is a peptide.

The terms “peptide” and “polypeptide” are used herein in their broadest sense to refer to a molecule of two or more amino acid residues, or amino acid analogs. The amino acid residues may be linked by peptide bonds, or alternatively by other bonds, e.g., ester, ether etc., but in most cases will be linked by peptide bonds.

As used herein, the terms “amino acid” or “amino acid residue” encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine.

Suitable methods of preparing a peptide, as herein described, would be familiar to persons skilled in the art. An illustrative example of which includes peptide synthesis that involves the sequential formation of peptide bonds linking each peptide sequence, and recovering said peptide. Other illustrative examples include the methods described in “Amino Acid and Peptide Synthesis” (Oxford Chemistry Primers; by John Jones, Oxford University Press). Synthetic peptides can also be made by liquid-phase synthesis or solid-phase peptide synthesis (SPPS) on different solid supports (e.g. polystyrene, polyamide, or PEG). SPPS may incorporate the use of F-moc (9H-fluoren-9-ylmethoxycarbonyl) or t-Boc (tert-Butoxycarbonyl). Custom peptides are also available from a number of commercial manufacturers.

Alternatively, the peptide may be prepared by recombinant methodology. For example, a nucleic acid molecule comprising a nucleic acid sequence encoding the fusion protein can be transfecting into a suitable host cell capable of expressing said nucleic acid sequence, incubating said host cell under conditions suitable for the expression of said nucleic acid sequence, and recovering said fusion protein. Suitable methods for preparing a nucleic acid molecule encoding the fusion protein will also be known to persons skilled in the art, based on knowledge of the genetic code, possibly including optimising codons based on the nature of the host cell (e.g., a microorganism) to be used for expressing and/or secreting the recombinant fusion protein. Suitable host cells will also be known to persons skilled in the art, illustrative examples of which include prokaryotic cells (e.g., E. coli) and eukaryotic cells (e.g., P. pastoris).

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated, typically in a host cell, to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product. In some embodiments, the nucleic acid sequence encoding the peptide sequences, as herein described, are codon-optimised for expression in a suitable host cell. For example, where the peptide is to be used for suppressing an immune response in a human subject, the nucleic acid sequences can be human codon-optimised. Suitable methods for codon optimisation would be known to persons skilled in the art, such as using the “Reverse Translation” option of “Gene Design” tool located in “Software Tools” on the John Hopkins University Build a Genome website.

Suitable peptides will be known to persons skilled in the art, illustrative examples of which include, for example, peptides derived from CD36 and CD36L1.

In an embodiment, the peptide is selected from the group consisting of CD36 and CD36L1.

In an embodiment, the ligand is a non-peptide molecule.

The term “non-peptide molecule” as used herein encompass numerous chemical classes, though typically they are organic molecules, preferably small organic compounds having a molecular weight of more than 50 and less than about 2,500 Dalton. Such molecules comprise functional groups necessary for structural interaction with proteins, particularly hydrogen bonding, and typically include at least an amine, carbonyl, hydroxyl or carboxyl group, desirably at least two of the functional chemical groups and often comprises cyclical carbon or heterocyclic structures or aromatic or polyaromatic structures substituted with one or more of the above functional groups. Suitable non-peptide agents would be known to persons skilled in the art, illustrative examples of which include biomolecules including, but not limited to saccharides, fatty acids, lipids, steroids, purines, pyrimidines, derivatives, structural analogs or combinations thereof.

Small (non-peptide) molecule ligands are particularly advantageous. In this regard, small molecules are desirable because such molecules may be more readily absorbed after administration, have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

In another embodiment, the ligand is a soluble ligand.

In another embodiment, the ligand is a soluble CD1 agonist.

The ligand described herein may be formulated in a pharmaceutical composition further comprising one or more pharmaceutically acceptable carriers, diluents or excipients. The term “pharmaceutically acceptable” refers to physiologically and pharmaceutically acceptable forms of carriers, diluents or excipients.

As used herein, the term “effective amount” includes a non-toxic but sufficient amount of a CD1 agonist that is effective for activating signalling through a CD1 molecule expressed by an APC in the subject. The effective amount required will vary from subject to subject depending on one or more relevant factors, illustrative examples of which include the species of the subject to be treated, the age and general health and wellbeing of the subject and the mode of administration. For any given case, an appropriate “effective amount” may be determined by one of ordinary skill in the art using only routine experimentation. For example, a ligand such as an antibody or antigen-binding fragment thereof may be administered in amounts of from about 50 μg to about 2,000 mg (e.g., including 100 μg, 200 μg, 300 μg, 500 μg, 800 μg, 1,000 μg, 10 mg, 20 mg, 50 mg, 100 mg, 500 mg, 1,000 mg, 1,500 mg and 2,000 mg or an amount in between). Alternatively, the antibody may be administered at a rate of from about 0.5 μg to about 20 mg/kg (e.g., 0.5 μg, 1 μg, 10 μg, 100 μg, 1 mg, 10 mg or 20 mg/kg) by body weight every from about 1 hour to up to about 50 hours (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 hours). Useful times include from about 6 hours to about 24 hours (e.g., 6, 7, 8, 9, 10, 11, 12, 13, 14, 15, 16, 17, 18, 19, 20, 21, 22, 23, or 24 hours). Useful times are between from about 12 to about 24 hours (e.g., 12, 13, 14, 15, 16, 17, 18, 19, 20, 21, 22, 23 or 24 hours). Dosing of the ligand can be determined by the attending physician in accordance with dosing rates in practice. The administration amounts may be varied if administration is conducted more or less frequently, such as by continuous infusion, by regular dose every few minutes (e.g., 1, 2, 3 or 4 minutes) or by administration every 5, 10, 20, 30 or 40 minutes (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39 or 40 minutes) or every 1, 2, 3, 4, 5, 6, 7, 8, 9, 10, 11, 12, 13, 14, 15, 16, 17, 18, 19, 20, 21, 22, 23 or 24 hours or up to 50 hours (e.g., 25, 26, 27, 28, 29, 30, 31, 32, 33, 34, 35, 36, 37, 38, 39, 40, 41, 42, 43, 44, 45, 46, 47, 48, 49 or 50 hours). In many instances, administration is conducted simply on the basis of when the patient requires suppression of an immune response.

A further aspect also provides a composition comprising a ligand that activates signalling through a CD1 molecule expressed by an APC, together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject.

In an embodiment, the composition further comprises a pharmaceutically acceptable carrier. In another embodiment, the pharmaceutically acceptable carrier is selected from the group consisting of a cell, a membrane, a polymer and an insoluble carrier.

In an embodiment, the composition further comprises an agent that facilitates signalling through the CD1 molecule expressed by an APC, an illustrative example of which includes an anti-Ig antibody to crosslink the primary anti-CD1 antibodies.

In another embodiment, the composition is co-administered with an agent that facilitates signalling through the CD1 molecule expressed by an APC.

The term “co-administration” as used herein includes both simultaneous and sequential administration. Simultaneous administration includes where the composition and the agent are present in two different formulations, but each are nevertheless administered at substantially the same time. Alternatively, the composition and the agent may be present in the same formulation.

In the case of sequential administration, a multi-step procedure is used where the composition is administered in one step and the agent is administered as a different time in a separate step. The composition may be administered prior to the administration of the agent, or visa versa. The time difference between administration of the composition and the agent can vary, but will generally range from about 1 minute to about 4 days, for example from about 1 minute to about 2 hours, or from about 1 minute to about 24 hours, or from about 1 minute to about 12 hours, or from about 1 minute to about 6 hours, or from about 1 minute to about 3 hours, or from about 1 minute to about 1 hour.

The composition may be conveniently presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions disclosed herein that are suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g., inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e., transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds described herein may also be presented as implants, which may comprise a drug bearing polymeric device wherein the polymer is biocompatible and non-toxic. Suitable polymers may include hydrogels, silicones, polyethylenes and biodegradable polymers.

The composition may be administered in a sustained (i.e., controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, the composition may be formulated with one or more additional immunosuppressive mediators such as prednisone, dexamethasone, hydrocortisone, cyclophosphamide, methotrexate, azathioprine or a pharmaceutical salt, derivative, homolog or analog thereof. The composition may be sold or provided with a set of instructions in the form of a therapeutic protocol. This protocol may also include, in one embodiment, a selection process for type of patient or type of condition or type of autoimmune disorder.

The present disclosure further contemplates nanoparticulate formulations that include nanocapsules, nanoparticles, microparticles, liposomes, nanospheres, microspheres, lipid particles, and the like. Such formulations increase the delivery efficacy and bioavailability and reduce the time for immunosuppression to occur. Nanoparticles generally comprise forms of the agents entrapped within a polymeric framework or other suitable matrix. Nanoparticle formulations are particularly useful for sparingly water soluble drugs. Such formulations also increase bioavailability. One method of formulation is a wet bead milling coupled to a spray granulation.

It should be understood that in addition to the ingredients particularly mentioned above, the compositions herein may include other agents conventional in the art, having regard to the type of composition in question. For example, agents suitable for oral administration may include such further agents as binders, sweeteners, thickeners, flavouring agents, disintegrating agents, coating agents, preservatives, lubricants and/or time delay agents.

The composition may also contain carriers, diluents and excipients. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

Also taught herein is a pharmaceutical composition comprising a ligand that activates signalling through a CD1 molecule expressed by an APC for use in a method of suppressing the immune system in a subject in need thereof.

Further taught herein is a pharmaceutical composition comprising a ligand that activates signalling through a CD1 molecule expressed by an APC for use in the treatment of an autoimmune disease, an inflammatory disease, or allergy in a subject. In an embodiment, the inflammatory disease is transplant rejection or graft-versus-host disease.

Also taught herein is the use of a pharmaceutical composition comprising a ligand that activates signalling through a CD1 molecule expressed by an APC in the manufacture of a medicament for the treatment of an autoimmune disease, an inflammatory disease or allergy. In an embodiment, the inflammatory disease is transplant rejection or graft-versus-host disease.

Further taught herein is a composition comprising a ligand that activates signalling through a CD1 molecule expressed by an APC, or other CD1 expressing cell type such as an epithelial cell, for use in inducing an immunosuppressive phenotype in an APC.

The present disclosure also contemplates methods for suppressing an immune response in a subject by administering APCs that have been modified in vitro to induce an immunosuppressive phenotype. Accordingly, in an aspect taught herein there is provided a method for suppressing an immune response in a subject, the method comprising:
d. isolating APCs from the peripheral blood of a donor;
e. culturing the isolated APCs with a composition comprising a ligand that activates signalling through a CD1 molecule expressed on the APCs for a time and under conditions suitable to induce an immunosuppressive phenotype in the APCs; and
f. administering the APCs with an immunosuppressive phenotype to the subject, in an amount sufficient to suppress the immune response in the subject.

Typically, the administration of cell therapies (e.g., APCs with an immunosuppressive phenotype) is defined by the number of cells per kilogram of body weight. In an embodiment, the APCs with an immunosuppressive phenotype of the present disclosure may be administered at a dosage of from about 10⁴ to about 10⁹ cells/kg body weight. In another embodiment, the APCs with an immunosuppressive phenotype of the present disclosure may be administered at a dosage of from about 10⁵ to about 10⁶ cells/kg body weight, including all integer values within those ranges.

In an embodiment, the APCs are dendritic cells. In another embodiment, the APCs are monocyte-derived dendritic cells (Mo-DCs).

In an embodiment, the subject is a transplant recipient.

The term “autologous” refers to any material derived from the same individual to whom the material is later to be re-introduced to the individual.

The term “allogenic” refers to any material derived from a different individual of the same species as the individual to whom the material is introduced. Two or more individuals are said to be allogenic to one another when the genes at one or more loci are not identical. In some aspects, allogenic materials from individuals of the same species may be sufficiently genetically distinct to interact antigenically.

The present disclosure contemplates the use of both autologous APCs and allogenic APCs in the methods disclosed herein. In a preferred embodiment, however, the donor is also the transplant recipient (i.e., autologous APCs).

In an embodiment, APCs are isolated from the subject prior to the administration of the transplant. In another embodiment, the APCs with an immunosuppressive phenotype are administered to the subject after administration of the transplant.

In another aspect disclosed herein, there is provided a method for identifying CD1-restricted T cells from a plurality of immune cells, the method comprising:
a. obtaining a plurality of immune cells from the peripheral blood of a donor;
b. contacting the plurality of immune cells with a composition comprising a first ligand, wherein the first ligand blocks the interaction between a CD1 molecule and a CD36 family member;
c. contacting the plurality of immune cells with second ligand, wherein the second ligand is capable of binding a CD1-restricted TCR; and
d. identifying CD1-restricted cells, wherein the CD1-restricted cells comprise second ligand bound to the cell surface.

In an embodiment, the first ligand is an antibody or an antigen-binding fragment thereof.

In an embodiment, the first ligand is an antibody to a CD36 family member or an antigen-binding fragment thereof.

In an embodiment, the second ligand is a CD1 multimer. Suitable CD1 multimers would be known to persons skilled in the art, illustrative examples of which include a CD1 dimer, CD1 trimer, CD1 tetramer, CD1 pentamer or higher order multimer.

In an embodiment, the second ligand is a CD1 tetramer.

In an embodiment, the CD1 tetramer is a human CD1 tetramer.

In an embodiment, the CD1 tetramer is selected from the group consisting of a CD1a, a CD1b, a CD1c and a CD1d tetramer.

The term “CD1 tetramer” as used herein refers to tetramised monomers loaded with lipid that is capable of binding to a CD1-autoreactive TCR. For example, the CD1 tetramers contemplated herein comprise recombinant CD1 monomers that are tetramerised using, e.g., fluorophore-conjugated streptavidin, as previously described by Kasmar et al., 2011, The Journal of Experimental Medicine, 208: 1741-1747), and loaded with lipids (e.g., self-lipids or lipid antigens) that allow them to bind to TCR of CD1-restricted T cells.

In an embodiment, the CD1 tetramer comprises a detectable label. In an embodiment, the detectable label is a fluorophore. In another embodiment, the fluorophore is BV421.

In an embodiment, the CD1 tetramer is loaded with lipids that are presented by CD1 isoforms. Suitable lipids would be known to persons skilled in the art, illustrative examples of which include phospholipid (e.g., phosphatidylcholine (PC)), oxidized phospholipid (e.g., lysophoshatidycholine (LPC), sulfoglycolipid (e.g., sulfatide), and GD3 ganglioside.

CD1-restricted cells would be known to persons skilled in the art, illustrative examples of which include of CD1d-restricted NKT cells, CD1b-restricted T cells (e.g., GMM-reactive GEM T cells, TRAV1-2+ GEM T cells), CD1a restricted T cells and CD1c restricted T cells.

The skilled person would appreciate that the contacting of the plurality of immune cells with labelled CD1 tetramer in accordance with step (c) may be variously described as a “tetramer assay,” “tetramer staining” or “multimer staining.”

In an embodiment, the CD1-restricted cells are identified using flow cytometry. Methods for the identification of cells using flow cytometry devices and systems would be well known to persons skilled in the art, illustrative examples of which include fluorescence-activated cell sorting (FACS).

In an embodiment, the method for identifying CD1-restricted T cells from a plurality of immune cells further comprises separating the CD1-restricted cells identified in step (d) from the plurality of immune cells, thereby isolating the CD1-restricted cells.

In an embodiment, the method for identifying CD1-restricted T cells from a plurality of immune cells is an ex vivo method.

### EXAMPLES

The present invention is now described by the following non-limiting Examples.

### Methods

### Human Samples

Healthy human blood buffy coats were obtained from the Australian Red Cross. PBMCs were isolated by standard density gradient (Ficoll-Paque Plus, GE Healthcare Life Science) and either used fresh or cryopreserved for subsequent analysis.

### Human PBMC Analysis:

For direct ex-vivo analysis, human PBMCs were stained in PBS with LIVE/DEAD Fixable Far Red (Thermo Fisher Scientific) for 15 mins at RT. Cells were then blocked with human Fc-block (BD Biosciences) in the presence or absence of purified anti-CD36 (Biolegend) for a further 15 mins at room temperature. Cells were washed once and stained in PBS+2% v/v FBS for 30 mins at room temperature with surface mAb anti-CD3ε, -CD14, -CD19, -CD42b -CD36, -CD36L1 (all from BD Pharmingen), and human CD1 tetramers (streptavidin-PE from BD Pharmingen). Cells were washed twice and subsequently fixed with 2% paraformaldehyde in PBS for 10 mins at room temperature. Cells were acquired immediately by flow cytometry using an LSRFortessa analyser (BD Biosciences) and subsequently analysed using Flowjo software (Treestar).

### CRISPR Library Screen

CRISPR library screening was performed as per previously described (Shalem et al. (2014, Science, 343(6166): 84-87). In brief, the human GeCKO v1 sgRNA library was lentivirally transduced into C1R cells in three biological replicate infections. Transduced cells were selected by puromycin. Each infection pool was then stained with CD1c tetramers and tetramer-negative cells enriched using FACS. Enrichment was performed twice to enrich for C1R cells containing sgRNAs that result in loss of tetramer staining. Genomic DNA was then extracted from pre- and post-enrichment samples of each pool. sgRNAs were subjected to 2 rounds of PCR, the first to amplify sgRNAs, the second to attach illumine adapters and barcode samples. Amplicons were then sequenced using HiSeq 2500 (Illumina).

### Generation of CRISPR/Cas9-Mediated Knockout Cell Lines

For CD36L1 knockout lines, lentiCRISPRv2GFP and lentiCRISPRv2mCherry plasmids were obtained from Addgene, and sgRNA cloning performed as described by the GeCKO Lentiviral CRISPR toolbox (Zheng Lab, Broad Institute, MIT), using the following oligos: 1. CACCGTCATGAAGGCACGTTCGCCG and 2. CAGTACTTCCGTGCAAGCGGCCAAA to generate a sgRNA specific for the target sequence CGGCGAACGTGCCTTCATGA within the CD36L1 gene. HEK293T cells were then transiently transfected with both GFP and mCherry vectors using Fugene HD reagent. After 3 days of transfection, cells were harvested and single cell sorted for high GFP and mCherry co-expression plus low CD36L1 expression. After in vitro propagation for 2-3 weeks, clones were screened for CD36L1 expression compared to wildtype HEK293T cells. One HEK293T.CD36L1-KO cell line was further propagated, and aliquots cryopreserved for subsequent experiments.

### Transient Transfections

Full-length CD36, CD36L1 (i.e., SCARB1) and CD36L2 (i.e., SCARB2) genes, as well as control TCRα- and β-chain genes linked by a p2A-linker were cloned into the pMIGII expression vector. Vectors were used to transiently transduce HEK293T cells using FUGENE (Promega). Control TCR transductions also included a pMIG vector encoding CD3ε, δ, γ and ζ. Cells. Cells were harvested after 48 hours, washed once in PBS+2% v/v FBS, then stained for 30 mins at RT with CD1 tetramers. Cells were then washed twice, fixed in 2% w/v PFA for 15 mins at RT before a final wash step. Cells were acquired immediately by flow cytometry using an LSRFortessa analyser (BD Biosciences) and subsequently analysed using Flowjo software (Treestar).

### CD1 Tetramers

Human CD1 tetramers were generated in-house as previously described by Birkinshaw et al., (2015, Nature Immunology, 16: 258-266); Van Rhijn et al., (2016, Proceedings of the National Academy of Sciences U.S.A, 113: 380), Wun et al., (2018, Nature Immunology, 19: 397) and Uldrich et al., (2013, Nature Immunology, 14: 1137). In brief, truncated CD1 extracellular domains containing an N-terminal 6×His-tag and thrombin cleavage site were cloned into a pHLSec mammalian expression vector. Vectors were co-transfected with a pHLSec vector encoding β₂-microglobulin into HEK293S.GNT1 cells using PEI transfection reagent. Expression supernatants were harvested and CD1 monomers purified by using Ni-NTA agarose (Thermo Fisher Scientific). Purified CD1 was biotinylated using Bir-A enzyme, and biotinylated monomers further purified with a superdex-200 10/300 GL column (GE) using size-exclusion chromatography. Purified biotinylated CD1 monomers were stored in aliquots at −80 C. Aliquots were tetramerized as required by five sequential additions of streptavidin-PE (BD Pharmingen) spaced every 5 mins at room temperature. Tetramers were then stored in the dark at 4° C. prior to use.

MR1-5-OP-RU tetramers were produced as previously described by Koay et al., (2019, Nature Communications, 10: 2243). In brief, truncated ectodomains of human MR1.C262S were expressed as inclusion bodies in Escherichia coli (strain BL21) along with human β2-microglobulin (β2m). MR1 and β2m inclusion bodies were then refolded in vitro in the presence of 5-A-RU and methylglyoxal using oxidative refolding, prior to dialysis and subsequent purification using Ni-NTA agarose. Human MR1-5-OP-RU were enzymatically biotinylated using BirA enzyme by further purification by size exclusion chromatography prior to storage at −80° C. All monomers were tetramerised using streptavidin-PE, -PE-Cy7 or -BV421 (all BD Pharmingen).

### Lipids

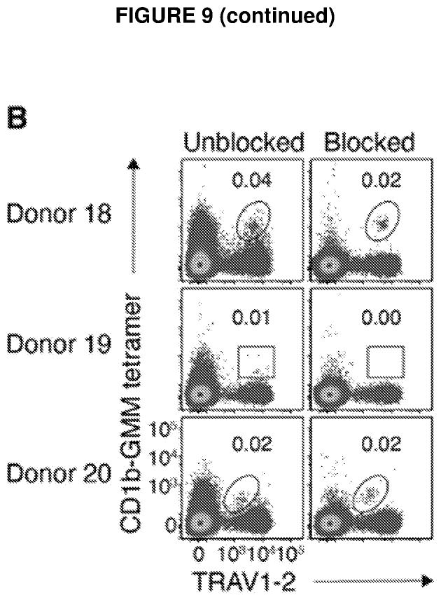

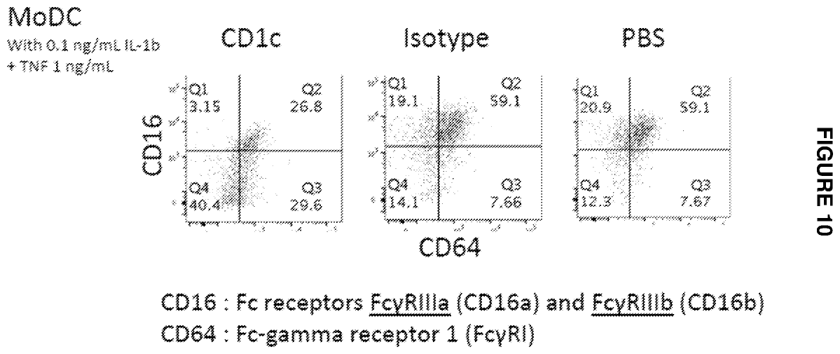

Lipids were dissolved by sonication in TBS (10 mM Tris pH 8.0, 150 mM NaCl) containing 0.05% (v/v) tyloxapol and stored at −20° C. prior to use. Prior to loading, lipids were thawed and re-sonicated for 30 min. All loading was performed overnight at RT. For staining, CD1d was loaded with α-GalCer (KRN7000) at a 3:1 molar ratio (lipid:CD1). For FIG. 9, CD1d was loaded with PBS44 at 6:1, LPC (C18:1) or sulfatide (C24:1), both at 12:1. CD1c was loaded with LPC (C18:1), sulfatide (C24:1), PC (C18:0-C18-1) or GD3 at 12:1. KRN7000, LPC, PC, sulfatide and GD3 were purchased from Avanti Polar Lipids. CD1b was loaded with GMM at 6:1.

### Mo-DC Preparation

PBMCs were isolated from healthy donors by density standard gradient centrifugation (Ficoll-Paque, GE Healthcare Life Sciences). Monocytes were enriched by anti-CD14 microbead separation (Miltenyi). Monocyte-derived dendritic cells were generated by culturing CD14-enriched PBMCs in 1,500 μl tissue culture media (RPMI-1640 supplemented with 10% fetal bovine serum (v/v), 15 mM HEPES (Gibco), 1 mM sodium pyruvate (Gibco), 0.1 nM non-essential amino acids (Gibco), 50 nM β-mercaptoethanol (Sigma), 100 U/ml penicillin (Invitrogen), 100 μg/ml streptomycin (Invitrogen) and 2 mM L-glutamine (Invitrogen)) containing recombinant human (Biolegend) GM-CSF (100 ng/ml) and IL-4 (50 ng/ml). Cells were seeded at 2.0×10⁶ cells per well in 24-well flat-bottom plates. After 48 hrs, cells were removed and centrifuged at 200×g for 5 min, then re-seeded with 1,500 μl of RPMI-1640 tissue culture media containing GM-CSF (100 ng/ml) and IL-4 (50 ng/ml). Cells were then cultured for an additional 48 hrs prior to harvest.

### CD1 Cross-Linking Assay

96-well flat-bottom plates were coated with 10 μg/ml functional grade antibody (<0.01 EU/μg BioLegend) diluted in 100 μl phosphate buffered saline (PBS) (Gibco) for 1 hr at 37° C., 5% CO₂. For conditions using LPS as a form of co-stimulation, PBS was spiked with 1 ng/ml LPS (E. coli 0127:B8 Sigma). The antibody solutions were then removed from wells and Mo-DCs were seeded at 5×10⁵ per well in 120 μl of tissue culture media. For experiments using cytokines as a form of co-stimulation rather than LPS, media was supplemented with recombinant human (BioLegend) IL-1β (100 pg/ml) and TNF (1 ng/ml). Mo-DCs were then incubated for 48 hrs at 37° C., 5% CO₂. 30 μl of culture supernatant was removed for cytokine analysis, then 150 μl of PBS supplemented with 0.5% FBS and 2 nM EDTA was added to each well followed by a 30 min incubation at 4° C. to facilitate removal of adherent cells prior to FACS analysis.

### Cytokine Analysis

Culture supernatants were analysed by CBA flex set for humans (BD Biosciences) according to the manufacturer's instructions.

### Example 1

### CD1c Tetramers Bind to Non-T Cells in Human Blood

### CD1c Tetramers Bind to Non-T Cells in Human Blood

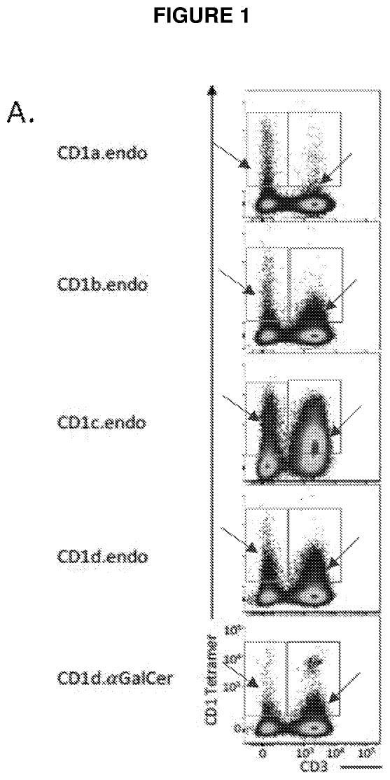

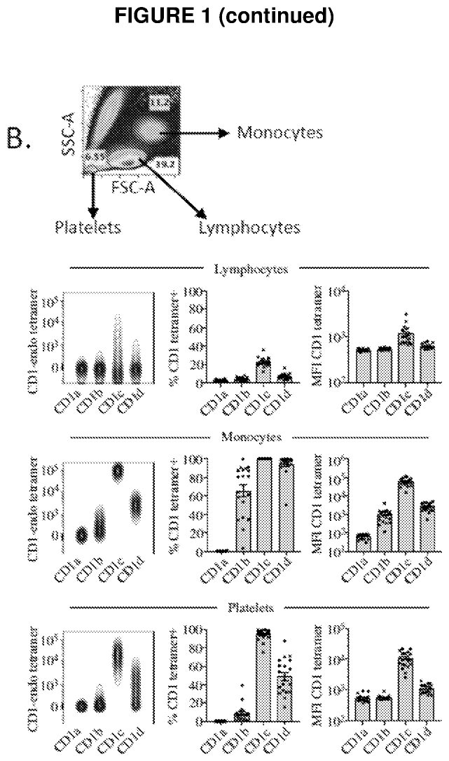

CD1 tetramers loaded with lipids are regularly used to identify CD1-restricted lipid-reactive T cells. A difficulty with such studies is that CD1-restricted T cells can be infrequent and difficult to detect in humans. In this study, a degree of what appears to be background staining with each of CD1a, CD1b, CD1c and CD1d tetramers was observed (FIG. 1A).

CD1c Tetramers Bind to Monocytes and Platelets from Human Blood

To further investigate the cause of this putative background staining, different populations of human blood cells were electronically separated into lymphocytes, monocytes and platelets and examined separately with each of the CD1 tetramers. This analysis revealed that CD1c tetramers bound strongly to CD3-negative CD14+ monocytes and platelets, CD1b and CD1d tetramers bound moderately to these cells, and CD1a tetramers showed less binding to these cells (FIG. 1B).

CD1c Tetramers Bind Via their α1/α2 Domains

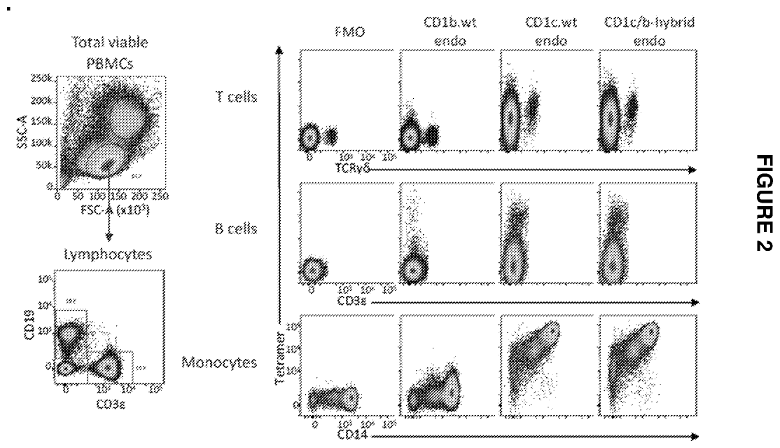

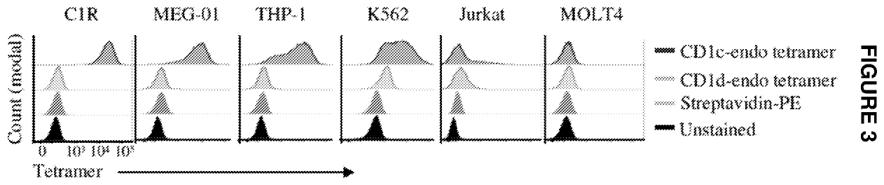

To determine whether CD1c tetramer binding was mediated by the α3 domain or the α1/α2 domains of the protein hybrid CD1c/CD1b tetramer was generated wherein the α1/α2 domains were from CD1c and the α3 domain was from CD1b (CD1b/c hybrid). These reagents were used to stain T cells, B cells and monocytes, which demonstrated that the α1/α2 domains of CD1c mediate binding to monocytes and also to some B cells (FIG. 2).

### CD1c Tetramers Bind to Non-T Cell Lines In Vitro

We also assessed the binding of CD1c tetramers in several other cell lines including C1R human B cell line, K562 human leukemia cell line, Jurkat human T cell line, and Molt4 human T cell line. CD1 tetramers bound to several different cell lines, including MEG01, C1R, Thp1 and to a lesser extent, K562 cells. As MEG01 and C1R are not T cells, a non-TCR ligand for CD1c appeared to be expressed by these cells.

### Identification of CD36 and CD36L1 as Ligands for CD1c

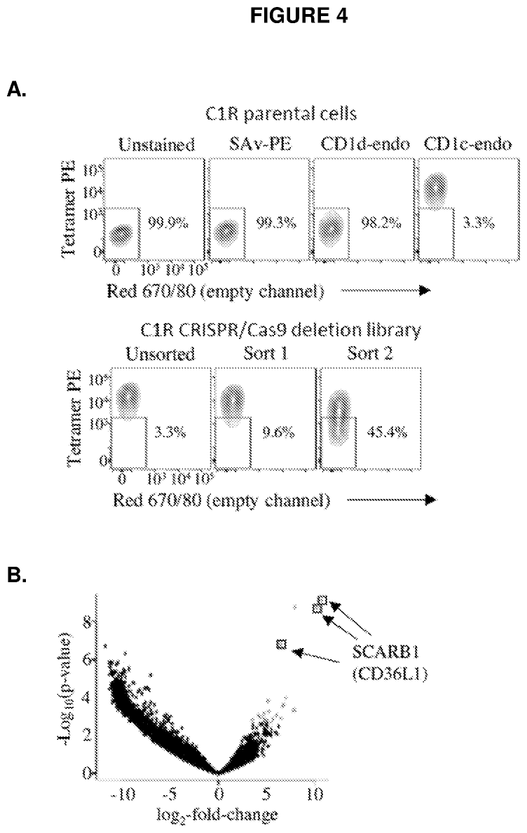

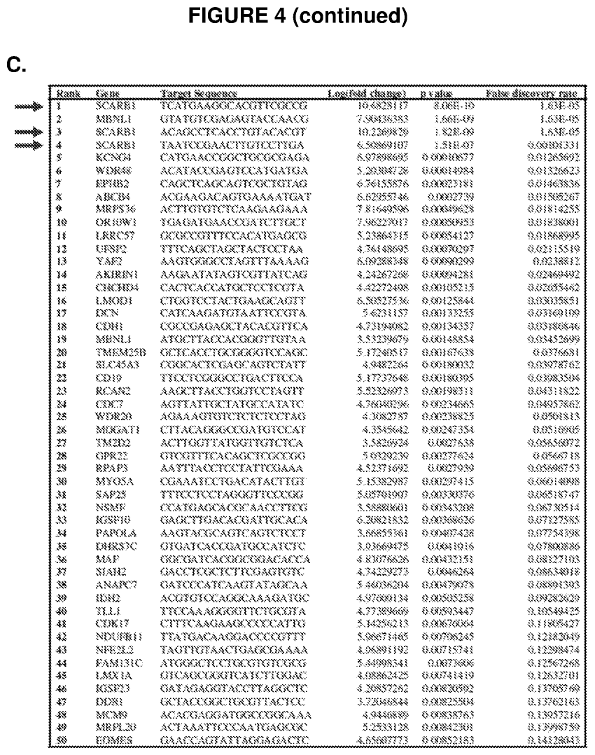

A CRISPR-Cas9 gene deletion library of C1R cells was used to investigate the ligand responsible for CD1c binding. After two rounds of sorting of CD1c tetramer^{low} CRISPR-Cas9-deleted C1R cells, a clear population of CD1c tetramer-negative cells was isolated (FIG. 4A). These CD1c tetramer-negative cells were compared to unselected C1R cells by comparing genes that were under-represented in the CD1c tetramer negative population, to determine which genes had been deleted that might be responsible for CD1c tetramer reactivity. This approach revealed four separate mutations, three of which were associated with a single gene, CD36L1 (also known as SCARB1 or SR-B1; FIG. 4B) and another mutation was associated with MBNL1, which is known to regulate alternative splicing of CD36L1 (FIG. 4C). CD36L1 is a scavenger receptor involved in cholesterol and lipid transfer (Shen et al., 2018, Annual Reviews Physiology, 80: 95-116), which was not previously recognised to be capable of binding to CD1 family members.

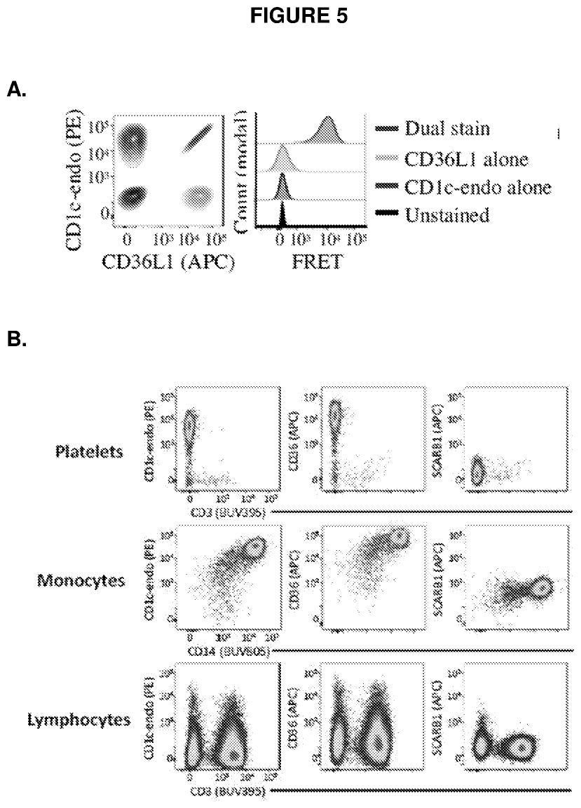

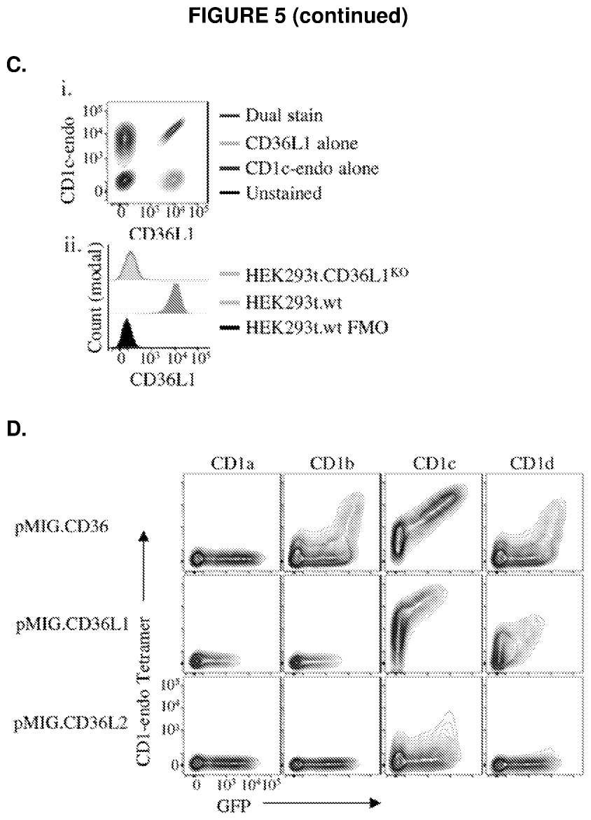

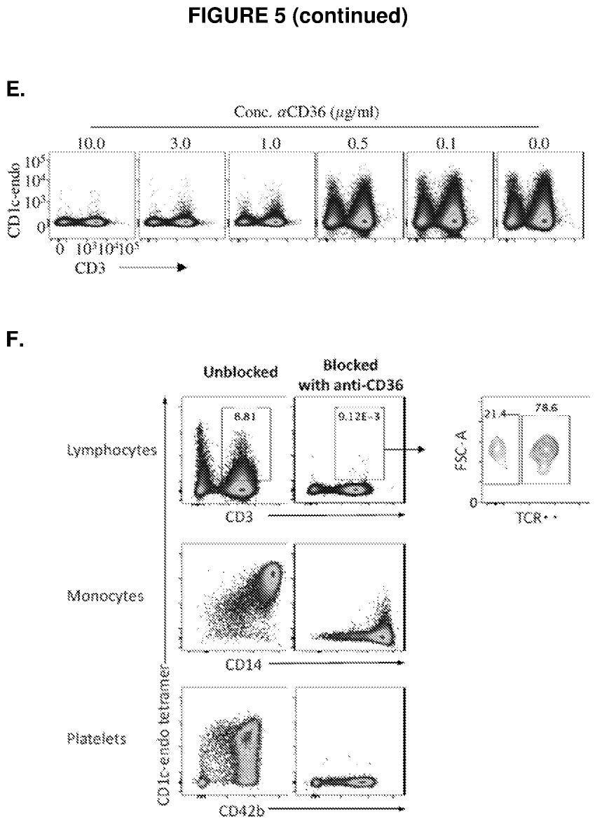

To further investigate a role for CD36L1 in binding to CD1c, C1R cells were co-labeled with CD1c tetramer and an anti-CD36L1 antibody (FIG. 5A). This experiment showed that parental C1R cells expressed high levels of CD36L1. Moreover, there was a direct correlation between CD36L1 and CD1c tetramer staining intensity, suggesting a direct association of these molecules on the cell surface. Furthermore, using a Förster Resonance Energy Transfer (FRET)-based approach to measure the proximity of CD36L1 and the CD1c tetramer, a strong FRET signal was detected, suggesting that the molecules were within 10 nm of each other and therefore closely associated on the cell membrane (FIG. 5A).

HEK293T cells (a human kidney cell line) were also stained with CD1c tetramer and this staining correlated directly with CD36L1 staining. To assess whether CD36L1 is sufficient for CD1c binding on HEK293T cells, CRISPR/Cas9 mediated gene deletion was used to generate a CD36L1 deleted HEK293T cell line. These CD36L1 knockout HEK293T cells lost all CD1c tetramer binding (FIG. 5C). Furthermore, the CD36L1 knockout HEK293T cell line, was reconstituted, by transfection, into subclones of these cells with CD36L1, CD36 (Febbraio et al., 2001, Journal of Clinical Investigation, 108: 785-791) or CD36L2. These data confirmed that CD1c was strongly reactive to CD36L1, but additionally CD1c tetramers were also shown to bind to CD36, and, to a lesser extent, CD36L2 transfected HEK293T cells (FIG. 5D). Interestingly, CD1d tetramer showed a similar pattern of reactivity, albeit with less intense staining, whereas CD1b tetramer bound to CD36 but not to CD36L1. Importantly, control HEK293T cell lines transfected with either CD1c- or CD1d-restricted TCRs stained only with CD1c or CD1d tetramers respectively. Taken together, it was demonstrated that CD1c and, to a lesser extent, CD1d, binds to CD36 and CD36L1.

### CD1c Staining of Human Monocytes and Platelets is Mediated by CD36 Expression

Human PBMC populations were tested for expression of CD36 and CD36L1 (SCARB1) and how this expression corresponded with CD1c tetramer staining (FIG. 5B). These data showed that CD36 was expressed by the vast majority of monocytes and platelets, and a subset (around 10-30%) of lymphocytes—an expression pattern that closely matched that of CD1c tetramer. In contrast, CD36L1 was not expressed by any of these PBMC populations, suggesting that CD36 was responsible for most of the CD1c tetramer reactivity to blood cells.

### CD36 Blockade Inhibits CD1b, CD1c and CD1d Tetramer Staining of Non-T Cells

PBMC populations were pre-incubated with a range of doses of blocking anti-CD36 antibody. At a concentration of 10 ug/ml of anti-CD36 antibody, CD1c tetramer staining of both CD3-positive and CD3-negative PBMC populations was inhibited (FIG. 5E). Additionally, pre-incubation of CD14-positive monocytes and CD42b-positive platelets with an anti-CD36 antibody blocked binding of CD1c tetramers to these cells (FIG. 5F). Importantly, anti-CD36 did not interfere with CD1b, CD1c and CD tetramer binding to CD1b, CD1c and CD1d restricted TCR+ cell lines which are expected to interact with CD1b, c and d tetramers, respectively, via TCR-mediated binding rather than CD36. These findings demonstrate that CD36 is responsible for non-TCR specific staining of CD1b, CD1c and CD1d tetramers and highlights the value of preincubating human PBMC with anti-CD36 antibody prior to CD1b, CD1c or CD1d tetramer staining in order to specifically detect CD1b, CD1c or CD1d restricted T cells.

### CD36 Blockade Facilitates Isolation of CD1-Restricted T Cells

PBMC from three donors were stained with CD1d-endo, CD1d-LPC and CD1d-sulfatide tetramers, using CD1d-αGalcer tetramers as a control (FIG. 9A). CD1d-endo and CD1d-LPC tetramers had substantial CD36-mediated staining; CD1d-endo and CD1d-LPC tetramers had substantial CD36-mediated staining on both T and non-T cells, whereas this was less prominent in the CD1d tetramers loaded with sulfatide or α-Galcer. Nonetheless, CD36-blockade prior to tetramer staining resulted in a marked reduction in non-TCR-mediated staining, revealing the rare residual population of tetramer⁺ T cells in each case. Thus, CD36-blockade facilitates the ex vivo analysis of CD1d-restricted T cells with lipid antigens, sulfatide, LPC and α-Galcer.

Using CD1b-GMM tetramers (FIG. 9B), it was also demonstrated that CD36-blockade facilitates the ex vivo analysis of CD1b-restricted T cells. For example, donors 18 and 20 were shown to have prominent populations of GEM T cells, which were more readily defined after CD36-blockade. Reciprocally, TRAV1-2+ cells that stained with CD1b-GMM tetramers in donor 19 were no longer present after CD36-blockade, reducing the false positive signal mediated by CD36.

These data demonstrate that pre-incubating PBMC with anti-CD36 antibody prior to CD1b, CD1c or CD1d tetramer staining facilitates the specific detection and isolation of CD1b-, CD1c- or CD1d-restricted T cells, particularly for populations of low frequency T cells where high signal:noise is required.

### Example 2

### Crosslinking of CD1a, CD1b and CD1c Drives PD-L1 and PD-L2 Upregulation and Immunoregulatory Cytokine Production

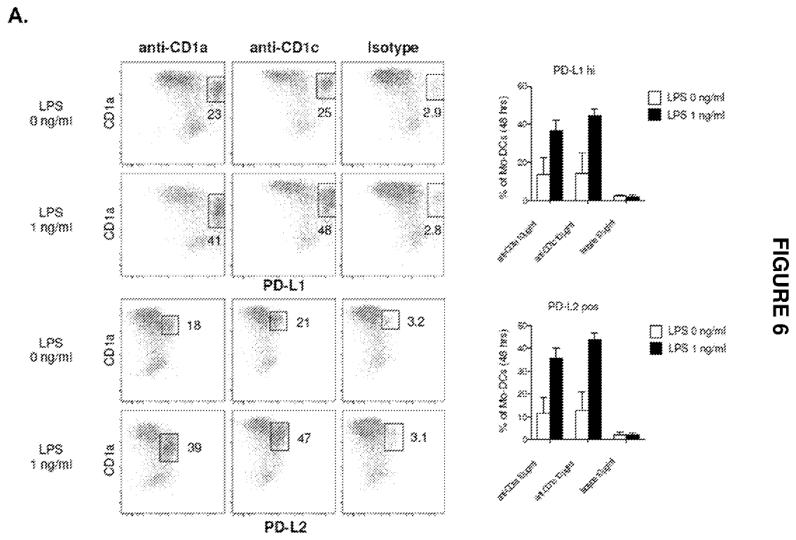

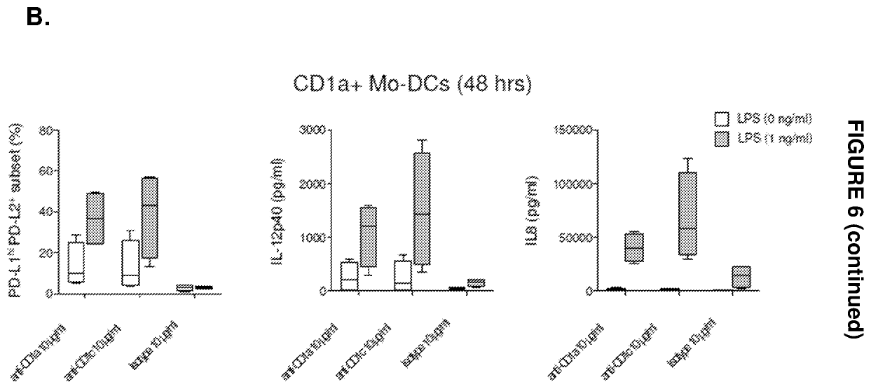

Human monocyte-derived dendritic cells (Mo-DC) were cultured in the presence of plate-bound anti-CD1a or anti-CD1c antibodies (designed to mimic engagement by a physiological ligand), or an isotype control antibody. These cells were found to upregulate programmed death ligand 1 (PD-L1) expression and programmed death ligand 2 (PD-L2) expression by a subset of Mo-DC, 2 days after their stimulation, in response to ligation with CD1a and CD1c (FIG. 6A). Furthermore, in the presence of a very low dose (1 ng/ml) of the exogenous inflammatory factor lipopolysaccharide (LPS) within the antibody solutions, anti-CD1a or anti-CD1c stimulation drove high levels of PD-L1 and PD-L2 expression that was not observed with the isotype control antibody (FIG. 6A). Multiparameter labelling demonstrated that the cells with high levels of PD-L1 were the same cells as those that had upregulated PD-L2.

### CD1a or CD1c Ligation Triggers IL-8 and IL-12p40 Cytokine Production by Mo-DC

Cytokine production was measured by Mo-DC following stimulation with anti-CD1a or anti-CD1c using cytometric bead array, focusing on cytokines that are known to be produced by myeloid cells. The samples stimulated by anti-CD1a and anti-CD1c selectively contained high levels of some cytokines, including interleukin-8 (IL-8) and IL-12p40 (FIG. 6B). The amounts of these cytokines were markedly enhanced by the presence of low dose LPS, whilst these cytokines were barely detectable in Mo-DC cultures incubated with an isotype control antibody in the presence or absence of LPS. IL-6 and low levels of IL-10 were also detected in these cultures but they appeared to be produced at similar levels in the presence of LPS plus isotype control antibody (data not shown). The production of IL-8 by Mo-DC is considered as an inhibitory factor in cell-mediated immune responses because it promotes angiogenesis and the recruitment of other inhibitory cells such as myeloid-derived suppressor cells (MDSC) (Alfaro et al., 2017, Cancer Treatment Review, 60: 24-31). The production of IL-12p40 may indicate the production of bioactive IL-12p70, IL-23 or IL-12p40 monomers or homodimers. However, given that IL-12p70 and IL-23 were not detected in these cultures (FIG. 6C), it appears that CD1 ligation induces production of IL-12p40 monomers or homodimers, which have potent immunosuppressive activity and can inhibit immune tumour immunity (Kundu et al., 2017, Proceedings of the National Academy of Sciences U.S.A, 114: 11482-11487). Lastly, a direct correlation between the induction of PD-L1/PD-L2 and the amounts of IL-12p40 and IL-8 in the cultures was observed, indicating that CD1 crosslinking induces a specific immunosuppressive population of MoDCs (FIG. 6D).

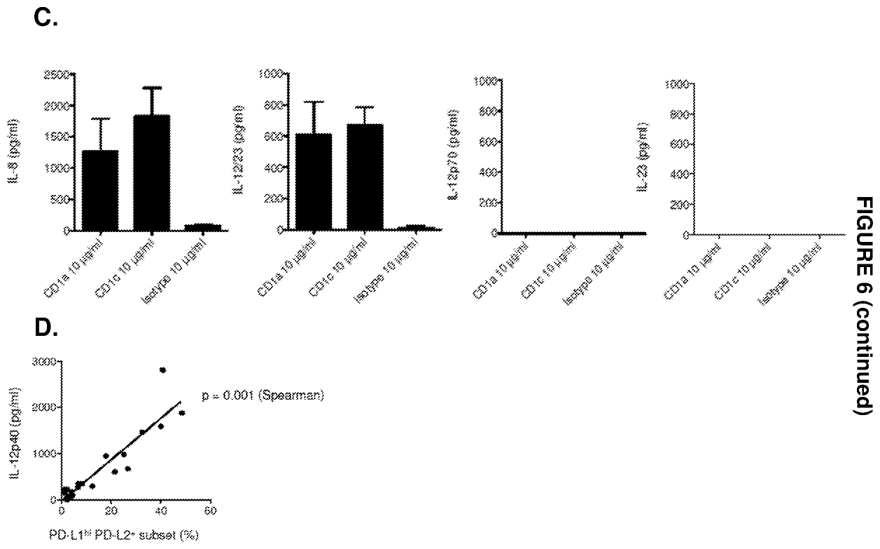

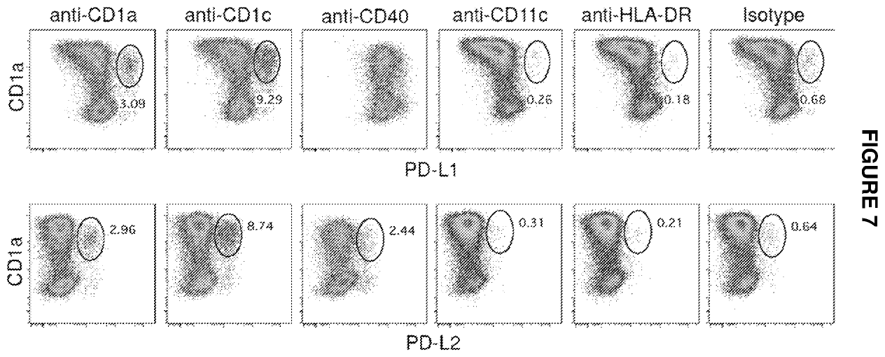

Given that crosslinking CD1a and CD1c gave similar responses in these assays, it was important to test the effect of crosslinking other unrelated molecules expressed on the surface of Mo-DCs. Thus, plate-bound antibodies against CD40, HLA-DR and CD11c were tested (FIG. 7). While no response was detected by crosslinking HLA-DR or CD11c, CD40 ligation induced PD-L1 upregulation by Mo-DCs. However, this response was clearly distinct from CD1a and CD1c crosslinking because in response to CD40 ligation, upregulation of PD-L2 was relatively low. Thus, CD1a or CD1c crosslinking appears to selectively promote an immunosuppressive Mo-DC phenotype leading to combined upregulation of PD-L1, PD-L2 cell surface expression and secretion of immunoregulatory cytokines IL-8 and IL-12p40.

### Cross-Linking Anti-CD1b Induces a PD-L1/PD-L2+ Phenotype on Mo-DC

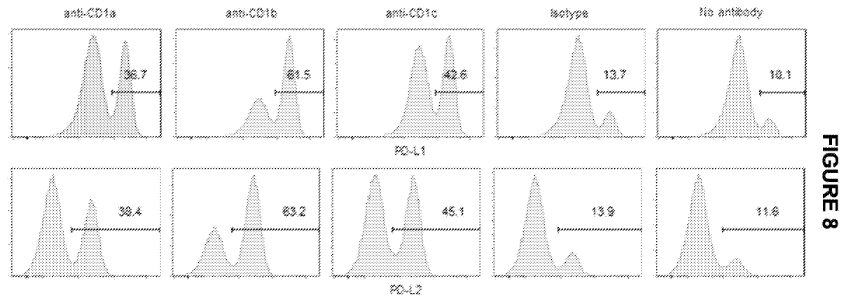

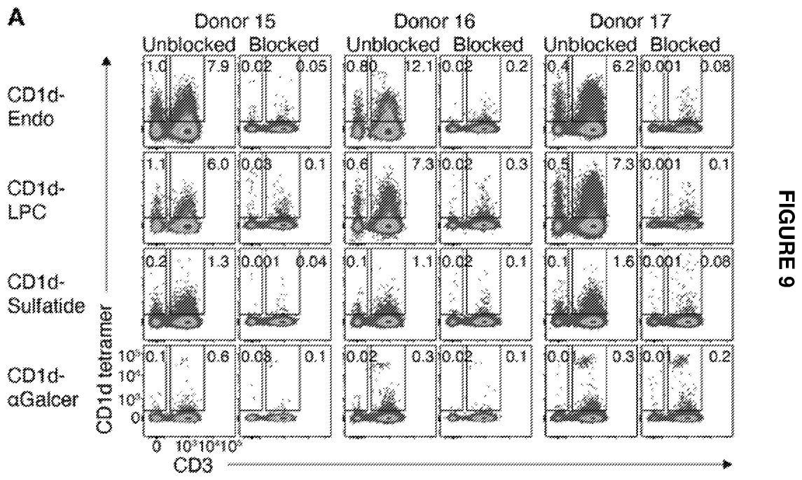

An anti-CD1b antibody was assessed for its ability to induce PD-L1 and PD-L2 expression by MoDC. In these cultures, IL-1β and TNF were used as a costimulatory factors instead of LPS. These data show clear upregulation of both PD-L1 and PD-L2 expression by Mo-DCs in the presence of anti-CD1b antibody, which was even stronger than the effects of CD1a and CD1c antibodies (FIG. 8).

### Example 3

### Crosslinking of CD1c Downregulates Fc Receptor Expression

CD16 (Fc receptors FcγRIIIa (CD16a), FcγRIIIb (CD16b)) and CD64 (Fcγ receptor 1 (FcγRI) play a critical role in dendritic cell (DC) maturation, DC cross-priming and presentation of CD8+ T cell responses to cancer, viral infection and autoimmunity (see, e.g., Platzer et al., 2014, Frontiers in Immunology, 5: 140).

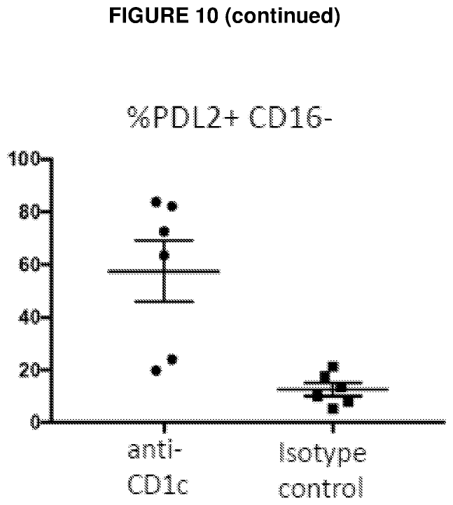

Following incubation with anti-CD1c antibody, it was shown that CD1 cross-linking downregulated Fc receptor expression on Mo-DCs (FIG. 10A) and increased the proportion of PD-L2+ CD16− Mo-DCs as compared to the isotype control (FIG. 10B). These data show clear upregulation of PD-L2 expression coincident with Fc receptor downregulation by Mo-DCs in the presence of anti-CD1c antibody, which further demonstrates that CD1 crosslinking appears to selectively promote an immunosuppressive Mo-DC phenotype leading to upregulation of PD-L2 cell surface expression and a corresponding downregulation of cell surface expression of Fc receptors.

Demonstrated herein is that Group 1 CD1 molecules, when cross-linked on the cell surface, are capable of transmitting signals to the cells that express them. This signaling leads to the induction of an immunosuppressive phenotype that includes upregulation of PD-L1 and PD-L2, production of immunoregulatory cytokines IL-12p40 and IL-8, and downregulation of cell surface expression of Fc receptors. Thus, it appears that CD1 crosslinking may have an important immunoregulatory function in the immune system. The results shown herein highlight the immunotherapeutic potential of targeting CD1 molecules with ligands that activate signalling through these molecules to suppress an immune response. For example, antibodies specific for CD1 family members will be expected to promote the immunosuppressive phenotype in APCs as observed in these in vitro studies of human cells. The effects of ligating these molecules was most prominent in an inflammatory setting (in the presence of LPS or inflammatory cytokines), suggesting that targeting these molecules would have the greatest impact in inflammatory diseases such as chronic infection, autoimmune diseases and allograft rejection.

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

The claims defining the invention are as follows:

1. A method for suppressing an immune response in a subject, said method comprising administering an effective amount of a composition comprising a ligand that activates signalling through a cluster of differentiation 1 (CD1) molecule expressed by an antigen presenting cell (APC) in the subject.

2. A method for inducing an immunosuppressive phenotype in an APC, said method comprising contacting the APC with an effective amount of a composition comprising a ligand that activates signalling through a CD1 molecule expressed by the APC.

3. The method of claim 1 or claim 2, wherein the CD1 molecule is a Group I CD1 molecule selected from the group consisting of CD1a, CD1b and CD1c.

4. The method of claim 3, wherein the Group I CD1 molecule is CD1c.

5. The method of claim 1 or claim 2, wherein the CD1 molecule is CD1d.

6. The method of any one of claims 1 to 5, wherein the ligand comprises a peptide, an antibody or antigen binding fragment thereof, or a non-peptide molecule.

7. The method of claim 6, wherein the ligand is an antibody or antigen binding fragment thereof.

8. The method of any one of claims 1 to 7, wherein the ligand is a soluble CD1 agonist.

9. The method of any one of claims 1 to 8, wherein the composition further comprises an agent that facilitates signalling through the CD1 molecule expressed by an APC.

10. The method of any one of claims 1 to 9, wherein the composition further comprises a pharmaceutically acceptable carrier.

11. The method of claim 10, wherein the pharmaceutically acceptable carrier is selected from the group consisting of a cell, a membrane, a polymer and an insoluble carrier.

12. The method of any one of claims 1 to 11, wherein the APC is a professional APC.

13. The method of claim 12, wherein the APC is a dendritic cell.

14. The method of any one of claims 1 to 13, wherein the activation of signalling through the CD1 molecule expressed by the APC in the subject is characterised by upregulation of the expression of one or more of the molecules selected from the group consisting of PD-L1, PD-L2, IL-12p40 and IL-8.

15. The method of any one of claims 1 to 14, wherein the subject has a condition selected from the group consisting of an autoimmune disease, an inflammatory disease, and allergy.

16. The method of claim 15, wherein the inflammatory disease is transplant rejection or graft-versus-host disease.

17. The method of any one of claims 1 to 15, wherein the subject is a transplant recipient.

18. A pharmaceutical composition comprising a ligand that activates signalling through a CD1 molecule expressed by an APC for use in a method of suppressing the immune system of a subject in need thereof.

19. Use of a pharmaceutical composition comprising a ligand that activates signalling through a CD1 molecule expressed by an APC in the manufacture of a medicament for the treatment of an autoimmune disease, an inflammatory disease, or allergy.

20. Use of claim 19, wherein the inflammatory disease is transplant rejection or graft-versus-host disease.

21. A pharmaceutical composition comprising a ligand that activates signalling through a CD1 molecule expressed by an APC for use in the treatment of an autoimmune disease, an inflammatory disease, or allergy in a subject.

22. The pharmaceutical composition of claim 21, wherein the inflammatory disease is transplant rejection or graft-versus-host disease.

23. A composition comprising a ligand that activates signalling through a CD1 molecule expressed by an APC for use in inducing an immunosuppressive phenotype in an APC.

24. A method for suppressing an immune response in a subject, the method comprising:
a. isolating APCs from the peripheral blood of a donor;
b. culturing the isolated APCs with composition comprising a ligand that activates signalling through a CD1 molecule expressed on the APCs for a time an under conditions suitable to induce an immunosuppressive phenotype in the APCs; and
c. administering the APCs with an immunosuppressive phenotype to the subject,
wherein the APCs with an immunosuppressive phenotype suppress the immune response in the subject.

25. The method of claim 24, wherein the APCs are dendritic cells or Mo-DCs.

26. The method of claim 24 or claim 25, wherein the subject is a transplant recipient.

27. The method of claim 26, wherein the donor is the transplant recipient.

28. The method of claim 27, wherein APCs are isolated from the subject prior to the administration of the transplant.

29. The method of any one of claims 26 to 28, wherein the APCs with an immunosuppressive phenotype are administered to the subject after administration of the transplant.

30. A method for identifying CD1-restricted T cells from a plurality of immune cells, the method comprising:
a. obtaining a plurality of immune cells from the peripheral blood of a donor;
b. contacting the plurality of immune cells with a composition comprising a first ligand, wherein the first ligand blocks the interaction between a CD1 molecule and a CD36 family member;
c. contacting the plurality of immune cells with second ligand, wherein the second ligand is capable of binding a CD1-restricted TCR; and
d. identifying CD1-restricted cells, wherein the CD1-restricted cells comprise second ligand bound to the cell surface.
